(19) United States
(12) Patent Application Publication
(10) Pub. No.: US 2003/0129748 A1
(43) Pub. Date: Jul. 10, 2003

(54) Enginerred bone marrow

(75) Inventors: Alan W. Flake, Bryn Mawr, PA (US); Alexander S. Krupnick, Philadelphia, PA (US)

Correspondence Address: FOLEY AND LARDNER, SUITE 500, 3000 K STREET NW, WASHINGTON, DC, 20007, US

(73) Assignee: Children's Hospital of Philadelphia

(21) Appl. No.: 10/280,056

(22) Filed: Oct. 25, 2002

Related U.S. Application Data
(60) Provisional application No. 60/330,658, filed on Oct. 26, 2001.

(51) Int. Cl. C12N 5/08

(52) U.S. Cl. 435/372

(57) ABSTRACT

An engineered bone marrow composition, preferably comprising bone marrow cells, pulverized bone, and type 1 collagen, can be transplanted into the portal system of a patient. The engineered bone marrow provides a microenvironment, for engraftment of hematopoietic stem cells, that increases levels of chimerism while decreasing or eliminating failure of engraftment.

The U.S. Government has a paid-up license in this invention and the right, in limited circumstances, to require the patent owner to license others on reasonable terms as provided for by provisions of a grant, entitled The Role of Mesenchymal Stem Cells in Fetal Tissue Engineering, that was awarded to N. S. Adzick, A. W. Flake and A. S. Krupnick by The Center for Innovative Minimally Invasive Therapy, funded by the Department of Defense.

### FIELD OF THE INVENTION

The present invention is directed to an artificial bone marrow, and to methodologies for making and using such a composition.

### BACKGROUND OF THE INVENTION

Bone marrow is a vital organ containing the cells that form both the host hematopoietic and immune systems. The production of blood cells, or hematopoiesis, takes place in the bone marrow. Hematopoiesis begins with a pluripotent hematopoietic stem cell (“HSC”), the class of which accounts for less than one per 10,000 nucleated bone marrow cells. The stem cell can either replicate and remain a stem cell or differentiate into a myeloid or lymphoid stem cell. The myeloid and lymphoid stem cells in turn can further proliferate and mature, ultimately giving rise to all circulating blood cells. Each of these complex hematopoietic pathways is under the influence of one or more hematopoietic growth factors or other cytokines that enhance cellular proliferation and maturation, as well as inhibitory activities that prevent proliferation. Bone marrow stromal cells that are in close contact with HSC provide the factors and cytokines that are required for the maintenance, replication, and differentiation of HSC. The close association required for the normal function of bone marrow is provided by a precise three-dimensional structure of the HSC and stromal cells that comprise the bone marrow microenvironment.

In the early 1950s, bone marrow transplants (BMT) were proposed for a number of diseases such as leukemia, lymphoma, anemia, erythrocyte disorders, inherited immune system disorders, metabolic diseases, and blood clotting disorders that were caused by various bone marrow defects. The BMT process involves the intravenous infusion of healthy pluripotent stem cells into a patient, relying on homing and engraftment of HSC within the host marrow. Bone marrow is not the only source of HSC, however. A small population of stem cells can be found in the peripheral circulation. The population of peripheral HSC can be increased by chemotherapy and harvested for transplantation. For this reason, the terminology has shifted from bone marrow transplant to hematopoietic stem cell transplant, HSCT. An HSCT is considered a success when donor HSCs are accepted by the patient and begin producing blood cells following HSCT.

The success of early HSCT experiments was limited due to the development of graft-versus-host disease, GVHD. GVHD is caused by an immune response between the immune cells derived from the host and donor HSC. In fact, early HSCT procedures in humans were only successful when donor cells from identical twins were used.

After these early HSCT procedures, the antigens responsible for promoting the immune response resulting in GVHD were identified. The genes for these antigens were found to reside in the chromosomal region known as the major histocompatibility complex, MHC. The MHC contains six genes that are closely linked and referred to as a haplotype. Each gene has multiple alleles resulting in a large number of possible MHC haplotypes.

A successful HSCT procedure requires a match between the haplotype of donor and host HSC. The HSCT procedure with the greatest likelihood of success is an autologous HSCT. In an autologous HSCT, host bone marrow cells are used for the transplantation procedure. While the rate of success is higher for autologous HSCT compared to other methods of transplantation, autologous HSCT is limited to the treatment of diseases not attributed to a defect in host HSC.

Allogeneic HSCT is recognized as the treatment of choice for a number of diseases caused by genetics defects in the host HSC population. In an allogeneic HSCT, a recipient receives HSC from a donor. Some of the disorders commonly treated with allogeneic HSCT are chronic myelogenous leukemia, acute leukemias failing initial treatment, and aplastic anemia. Furthermore, allogeneic bone marrow transplantation is increasingly used as a cure for genetic disorders associated with the hematopoietic and the immune systems, and for lipid storage diseases. Examples of the genetic diseases that have been cured by bone marrow transplantation are Cooley's anemia, sickle cell anemia, severe combined immunodeficiency, Wiskott-Aldrich syndrome, Fanconi anemia, Blackfan-Diamond anemia, ataxia telangiectasia, infantile agranulocytosis, Chediak-Higashi disease, chronic mucocutaneous candidiasis, mucopolysaccharidosis, cartilage-hair hypoplasia, Gaucher's and other lipid storage diseases. Some of these diseases, such as Cooley's anemia (beta-thalassemia) and sickle cell anemia, are major public health problems. Others are devastating orphan diseases that are extremely costly to treat. Each year tens-of-thousands of children are born with these genetic diseases.

The clinical success of HSCT is limited by the toxicity associated with many of the transplantation conditioning regiments. Postnatal HSCT has traditionally relied on a myeloablative conditioning method to create “space” within the bone marrow microenvironment. Recently, the medical community has challenged the need for these harsh preconditioning regimes. Less toxic non-myeloablative approaches for preparing a patient for HSCT are finding favor in the medical community.

One consequence of these milder HSCT preconditioning procedures is that the host bone marrow contains both the donor and host HSC, following HSCT. This situation is referred to as a “mixed hematopoietic chimerism.” When there is a perfect match between the HSC haplotype of the transplant donor and the transplant recipient, there is a high probability of engraftment following transplantation. Modern molecular techniques allow the haplotype of an individual to be precisely determined. This has resulted in the ability to match the haplotypes of a HSC recipient and an unrelated HSC donor and an increase in the success associated with HSCT. More often than not, medical personnel are unable to find donor HSC with the exact same MHC haplotype as the recipient.

In this situation, the ability to create a stable mixed hematopoietic chimerism is limited by inefficient engraftment or loss of engraftment due to GVHD. Currently, the only approach to increasing the efficiency of engraftment entails administration of immunosuppressive drugs.

Even with immunosuppression, though, there is experimental support for the observation that a MHC mismatch between donor stem cells and host microenvironment inhibits engraftment. One reason for this is competition between donor and host HSC. In one of the first reports examining the importance of the MHC of the microenvironment, bone grafts were transplanted into irradiated allogeneic recipients. In these experiments there was a preferential engraftment of MHC matched stem cells in the bone grafts following HSCT. In another study, engraftment of highly enriched HSC populations was improved by co-transplantation of MHC matched osteoblasts.

These studies suggest that a stable, mixed hematopoietic chimerism is facilitated by co-transplantation of HSC and a MHC matched bone marrow environment. Bone marrow, though, is a complex organ that contains hematopoietic cells and supporting stroma consisting of adventitial reticular cells, osteocytes, adipocytes, extracellular collagen matrix, and vascular endothelium. The close association of multiple cell types in a defined three dimensional organization is required for multilineage hematopoiesis. The close association of these cells requires a porous, three-dimensional lattice. The previous experiments do not suggest the components of bone marrow required for the formation of a HSC microenvironment necessary for efficient engraftment and multilineage hematopoiesis. Similarly, these experiments do not suggest the manner in which HSC and the cells that form the microenvironment can be combined to form an engineered bone marrow. Finally, these experiments do not suggest a transplantation method or methods that can be used to increase engraftment of an engineered bone marrow.

Because of the therapeutic potential of HSC transplants, there is a need in the art for an engineered bone marrow that recapitulates the three-dimensional structure of naturally occurring bone marrow. This engineered bone marrow must provide the structural elements and the cells needed to support engraftment and multilineage hematopoiesis. In addition, methods that increase the efficiency of engraftment are needed.

### SUMMARY OF THE INVENTION

Accordingly, the present invention addresses the need for an engineered bone marrow composition that has the essential morphological characteristics of native marrow and a methodology for transplanting the engineered bone marrow, in order to overcome the problems associated with hematopoietic stem cell transplantion.

In this regard, there has been provided, according to one aspect of the invention, an engineered bone marrow comprising bone marrow cells, pulverized bone or bone substitute, and type I collagen or collagen substitute. In a preferred embodiment, the engineered bone marrow is further comprised of a hematopoietic stem cell, wherein the bone marrow cells support the maintenance and differentiation of the hematopoietic stem cell, and wherein the hematopoietic stem cell and the bone marrow cells have the same MHC haplotype. In another preferred embodiment, the engineered bone marrow is comprised of the complete cellular component of bone marrow, obtained from a single donor.

In another embodiment, the engineered bone marrow is comprised of a bone substitute that is selected from the group consisting of a calcium phosphate, a bioactive glass, and a bioactive ceramic. In yet another embodiment, the engineered bone marrow is comprised of a collagen substitute that is a mammalian derived gelatin- or collagen-containing bioabsorbable sponge.

In one embodiment of the invention, the engineered bone marrow has a proportion of bone marrow cells: pulverized bone or bone substitute: collagen that is about 8-98%: 1-91%: and 0.004-1.0%. In a preferred embodiment, the proportion of bone marrow cells: pulverized bone or bone substitute: collagen is about 32-91%: 9-67%: 0.01-1.0%. In a more preferred embodiment, the proportion of bone marrow cells: pulverized bone or bone substitute: and collagen or collagen substitute is about 48-84%: 16-52%: 0.03-1.0%.

According to another aspect of the present invention, a methodology is provided for making an engineered bone marrow, comprising combining about 1×10⁶ to about 5×10⁸ bone marrow cells with about 50-500 mg of pulverized bone or bone substitute and about 100 μl to about 750 μl of neutralized type I collagen matrix that has a collagen concentration of about 1.0 mg/ml to about 2.0 mg/ml. A preferred embodiment comprises combining about 5×10⁶- to about 1×10⁸ bone marrow cells with about 50 mg to about 500 mg of pulverized bone or bone substitute and about 100 μl to about 750 μl of neutralized type I collagen matrix that has a collagen concentration of about 1.0 mg/ml to about 2.0 mg/ml. A more preferred embodiment comprises combining about 1×10⁷ to about 6×10⁷ bone marrow cells with about 50 to about 500 mg of pulverized bone or bone substitute and about 100 μl to about 750 μl of neutralized type I collagen matrix that has a collagen concentration of about 1.0 mg/ml to about 2.0 mg/ml. In a most preferred embodiment, 10×10⁶-20×10⁶ bone marrow cells are combined with 50 mg to about 200 mg of pulverized bone or bone substitute.

Yet another aspect of the invention provides a methodology for transplanting the engineered bone marrow. One embodiment concerns a method of implanting an engineered bone marrow, comprising introducing the engineered bone marrow of claim 1 into a vascular site in the portal system of a mammal. A preferred embodiment concerns a method wherein the engineered bone marrow is implanted in a pocket of an intramesenteric portal site. In a more preferred embodiment, the engineered bone marrow is implanted in an intramesenteric pocket of the small bowel mesentery.

In another embodiment, the ratio of the volume of the engineered bone marrow implanted to the volume of the mesentery is about 0.1 to about 0.75:1. In a more preferred embodiment, the ratio of the volume of engineered bone marrow implanted to the volume of the mesentery is about 0.1 to about 0.5:1.

Other features, objects, and advantages of the present invention are apparent in the detailed description that follows. It should be understood, however that the detailed description, while indicating certain aspects of the invention, are give by way of illustration only, and not limitation. Various changes and modifications within the spirit and scope of the invention will become apparent to those skilled in the art from the detailed description.

### DETAILED DESCRIPTION OF THE INVENTION

In studying the problems associated with hematopoietic stem cell transplantation, the inventors developed an engineered bone marrow that recapitulates the cellular composition and the three-dimensional structure required for HSC replication and multilineage hematopoiesis, as in normal bone marrow. Similarly, a methodology was established whereby the inventive composition, following transplantation in a mammal, increases the efficiency of HSC engraftment.

An engineered bone marrow of the invention has both a cellular component and structural components that provide the three-dimensional structure associated with normal bone marrow. The cellular component includes any type of cell normally found within bone marrow (“bone marrow cell”) and, optionally, any type of hematopoietic stem cell (HSC). Exemplary of the category of “bone marrow cell” are a stromal cell from bone morrow, a bone marrow stem cell, a fibroblast, an epithelial cell, an adipocyte, an osteocyte, an osteoblast, and a reticular cell.

In this context, “hematopoietic stem cell” is an unspecialized cell that can undergo differentiation into a specific cell type in response to environmental signals. Illustrative of a hematopoietic stem cell type is a bone marrow stem cell, a peripheral blood stem cell, and an umbilical cord stem cell, also known as a “fetal stem cell.”

Bone marrow stem cells are the most common type of HSC. Conventional methodology for isolating and maintaining bone marrow cells, prior to implantation, are described, for example, by Thomas et al., “Technique for human marrow grafting,” Blood 36: 507-15 (1970), Dexter et al., “Regulation of hematopoietic stem cell proliferation in long term bone marrow cultures,” Biomedicine 27: 344-49 (1977), and Pittenger et al. “Multilineage potential of adult human mesenchymal stem cells,” Science 284:143-7 (1999).

A peripheral blood stem cell (PBSC) is a stem cell that is found in the blood, albeit at a very low concentration.” In order to collect PBSCs, colony stimulating factor or growth factor is given to the donor to stimulate the bone marrow to produce more stem cells, which then are released into the blood stream. Once they are in the blood, PBSCs are collected in one or more sessions normally taking four to six hours each. In this procedure, called apheresis, the blood circulates through a machine called a cell separator, which removes the peripheral stem cells and returns the rest of the blood to the body. Methodology for isolating and preparing peripheral blood stem cells are described, for instance, by Majolino et al., “Peripheral blood stem cells for allogeneic transplantation. Recommendations from the GITMO 1996,” Haematologica 81:529-32 (1996), Novotny et al., “Sustained decrease of peripheral lymphocytes after allogeneic blood stem cell apheresis,” Br. J. Haematol. 100:695-7 (1998), and Dini et al., “Peripheral blood stem cell collection from G-CSF-stimulated unrelated donors for second transplant,” Bone Marrow Transplant 22(Suppl 5):S41-5 (1998).

The final type of HSC is the umbilical cord stem cell. Blood remaining in the placenta and umbilical cord of a newborn baby contains a large concentration of stem cells. The umbilical cord stem cells, instead of being discarded, may be harvested, and used for transplantation according to the methodology described by Cairo et al., “Placental and/or umbilical cord blood: an alternative source of hematopoietic stem cells for transplantation,” Blood 90:4665-4678 (1997), and Kurtzberg et al., “Placental blood as a source of hematopoietic stem cells for transplantation into unrelated recipients,” N Engl J Med. 335:157-166 (1996).

In a preferred embodiment, the engineered bone marrow is comprised of one or more bone marrow cell types that support the maintenance of HSCs and their differentiation, especially in terms of multilineage hematopoiesis. Examples of cell types involved in the differentiation of HSCs include osteoblasts, adipocytes, endothelial cells, bone marrow fibroblasts, bone marrow reticular cells, and bone marrow stromal cells. In order to facilitate the engraftment of the HSCs, the engineered bone marrow may be comprised of HSCs and one or more other bone marrow cell types that have the same MHC haplotype. In a preferred embodiment, the engineered bone marrow contains the complete cellular component of bone marrow derived from a single individual.

Pursuant to the present invention, the structural components of an engineered bone marrow include (i) pulverized bone or a bone substitute and (ii) collagen or a collagen substitute. Pulverized bone may be derived from any type of human or animal bone. Sources of sterile bone can be obtained commercially from a variety of sources, including but not limited to the Life Link Tissue Bank (Tampa, Fla.), Central Florida Tissue Bank (Orlando, Fla.), and the Pacific Coast Tissue Bank (Los Angeles, Calif.). For this invention, any one of a number of different methods can be used to produce pulverize bone. These procedures are carried out under sterile conditions. In a preferred embodiment, the pulverized bone has a small particle size, such that the pulverized bone can be passed through an 18-gauge needle, a size that allows the diffusion of nutrients to bone marrow cells.

An alternative to pulverized bone in this context is a bone substitute. Bone substitutes are described, for example, by Marshal et al., BONE GRAFTS, DERIVATIVES, AND SUBSTITUTES (Butterworth-Heinemann Lts., 1994). Calcium phosphates, such as hydroxyapatite, are generally considered materials of choice as bone substitutes. Low-density hydroxyapatite is a preferred calcium phosphate bone substitute due to its ability to form a bond with collagen. Examples of commercially available calcium phosphate bone substitutes are Hapex™, a product of Smith & Nephew Richards, Inc. (Memphis, Tenn.); Durapatite®, a product of Acti-Form LTD. (Markham, Ontario); and Calcitite®, which is manufactured by Calciteck Inc. (San Diego, Calif.).

In addition to calcium phosphates, there are a number of bioactive materials, such as glasses and ceramics, which may be used as bone substitutes. The common characteristic of these bioactive materials is that they are able to form a bond with bone or a soft tissue associated with bone such as collagen. In order to form a bond between tissues and the bioactive material, a layer of biologically active hydroxylcarbonate apatite (HCA) must form on the bioactive material following implantation.

In general, bioactive glasses and glass-ceramics require the combination of SiO₂, Na₂O, CaO, and P₂O₅ in specific proportions. There are three key compositional features to these bioactive glasses that distinguish them from traditional soda-lime-silica glasses: (1) they have less than 60 mole percent SiO₂, (2) they have a high Na₂O and CaO content, and (3) they have high CaO/P₂O₅ ratio.

In addition, many bioactive silica glasses are based upon the formula called “45S5”, signifying 45 wt % SiO₂ and a 5:1 molar ratio of Ca to P. Glasses with substantially lower molar ratios of Ca to P (in the form of CaO and P₂O₅) do not bond to bone. However, substitutions in the 45S5 formula of 5 to 15 wt % B₂O₃ for SiO₂ or 12.5 wt % CaF₂ for CaO or ceraming the various bioactive glass compositions to form glass-ceramics have no measurable effect on the ability of the material to form a bone bond. The addition of even small amounts of Al₂O₃, Ta₂O₅, TiO₂. Sb₂O₃, or ZrO₂ may inhibit the bonding to bone or tissue and, hence, generally are unsuitable as bone substitutes.

Illustrative bioactive glasses that are suitable as bone substitutes include: Bioglass®, NovaBone™, and NovaBone C/M™, all of which are products of US Biomaterials (Alachua, Fla.).

Similar to the bioactive glasses are the bioactive ceramics. It now is generally accepted that calcium-based ceramics may be used as bone substitutes. Both calcium sulphate- and calcium phospate-based ceramics have been employed in this regard. To date, the calcium phosphate based-ceramics, such as hydroxyapatite- and hydroxylapatite-based ceramics, that most closely resemble the inorganic constituent of bone are the most effective bone substitutes. Exemplary bioactive ceramics that can be used as bone substitutes are Ceravital®, Pro Osteon™ coralline hydroxyapatite ceramic, a product of Interpore International Company, (Irvine, Calif.), crystalline hydroxyapatite ceramic, which is available commercially from Zimmer Scientific (Warsaw, Ind.), and A/W glass ceramic.

The collagen that represents a second structural component of the invention is preferably type I collagen. Type 1 collagen is commercially available from several sources, including Sigma Chemical (St. Louis, Mo.) and Worthington Biochemical Corporation (Lakewood, N.J.). In lieu of collagen per se, one also can employ a mammalian derived gelatin- or a collagen-containing bioabsorbable sponge such as Gelfoam™, a product of Upjohn (Kalamazoo, Mich.), and Surgifoam™, a product of Ethicon (Somerville, N.J.). These products are commercially available and can be added directly to the mixture of bone marrow cells and pulverized bone or bone substitute, in the context of creating an engineered bone marrow of the present invention.

In order to prepare the engineered bone marrow of the present invention, bone marrow cells are combined with enough pulverized bone or bone substitute to provide a gelatinous composition. In one aspect of the invention, about 1×10⁶ to about 5×10⁸, about 5×10⁶ to about 1×10⁸, or about 1×10⁷ to about 6×10⁷ bone marrow cells are combined with about 50 mg to about 500 mg of pulverized bone or bone substitute. In another aspect of the invention, about 10×10⁶ to about 20×10⁶ bone marrow cells are combined with about 50 mg to about 200 mg of pulverized bone or bone substitute.

Next, sterile, collagen is added to the mixture of bone marrow cells and pulverized bone or bone-substitute. Collagen obtained commercially usually is acidified to prevent collagen fibers from crosslinking and forming a matrix. For the present invention, in contrast, the formation of a collagen matrix is desired. Accordingly, collagen that is added to a mixture of bone marrow cells and pulverized bone, according to the invention, preferably takes the form of a solution of neutralized collagen matrix, especially neutralized type I collagen matrix. Methods for producing neutralized type 1 collagen matrix solution are detailed, for example, by Eschenhagen et al., FASEB J. 11: 683-94 (1997), and Fink et al., loc. cit. 14: 669-79 (2000). The collagen concentration of the neutralized type 1 collagen matrix solution is about 1 mg/ml to about 2 mg/ml, preferably between about 1.3 mg/ml to about 1.5 mg/ml. In one embodiment, about 0.1 mg to about 1.5 mg of neutralized type 1 collagen matrix is added to the mixture of bone marrow cells and pulverized bone or bone substitute. In general, this requires an addition of about 100 μl to about 750 μl or preferably about 300 μl to about 500 μl of neutralized type I collagen matrix solution to the mixture of bone marrow cells and pulverized bone or bone substitute. In another embodiment, about 0.3 to about 1.0 mg of neutralized type 1 collagen matrix is added to the bone marrow composition.

The weight of the bone marrow cells can be determined by centrifuging the cells and removing any liquid. In general, 11-13×10⁶ bone marrow cell weigh about 53.5 to 65.8 mg, and an engineered bone marrow prepared using the above method has a weight ratio of about 8-98% bone marrow cells, about 1-91% pulverized bone or bone substitute, and about 0.004-1.0% of collagen or collagen substitute. In a preferred embodiment, the engineered bone marrow has a weight ratio of about 32% to about 91% bone marrow cells, about 9% to about 67% pulverized bone or bone substitute, and about 0.01% to 1% of collagen or collagen substitute. In a more preferred embodiment, the engineered bone marrow has a weight ratio between about 48 to about 84% bone marrow cells, about 16% to about 52% pulverized bone or bone substitute, and about 0.03 to about 1.0% of collagen or collagen substitute.

The inventors also have demonstrated that the transplantation site determines the efficiency of engraftment of the engineered bone marrow. The transplantation procedure requires implantation into a vascular tissue site, although not all vascular tissue sites allow efficient engraftment. Implantation of the inventive composition in a highly vascular intramuscular site, for instance, did not allow survival of the engineered bone marrow. By contrast, the inventors have found that implantation at a site in the portal system typically leads to the formation of a bone marrow type structure, in accordance with the present invention.

The portal system is that portion of the circulatory system that drains into the liver. Preferably, a composition of the invention is implanted in an intramesenteric portal site of the portal system, that is, in a portion of the portal system associated with the small or large intestine. To this end, an intramesenteric pocket is created by folding the leaves of the mesentery over one another in order to create space for engraftment.

A key consideration to successful clinical application of the inventive technology is the construction of an adequate volume of bone marrow, in order to support sufficiently high numbers of donor hematopoietic stem cells. For this purpose, the small bowel mesentery, with its large surface area, offers an ideal site to overcome space limitations.

In one aspect of the invention, the ratio of the volume of engineered bone marrow composition implanted to the volume of mesentery is about 0.1 to about 0.9 to 1. In a preferred embodiment, the ratio of the volume of engineered bone marrow composition implanted to the volume of mesentery is about 0.1 to about 0.75 to 1. In a more preferred embodiment the ratio of the volume of engineered bone marrow composition implanted to the volume of mesentery is about 0.1 to about 0.5 to 1.

The following examples are given to illustrate the present invention. It should be understood, however, that the invention is not to be limited to the specific conditions or details described in these examples.

### EXAMPLE 1

Cell Isolation: All animals were housed in the Laboratory Animal Facility of the Abramson Pediatric Research Center at the Children's Hospital of Philadelphia. All experimental protocols were reviewed and approved by the Institutional Animal Care and Use Committee at the Children's Hospital of Philadelphia, and followed guidelines set forth in the National Institutes of Health Guide for the Care and Use of Laboratory Animals.

Eight to twelve week old C57/BL6 female mice (Jackson Laboratories, Bar Harbor Me.) were used as the source of both bone and bone marrow. After sacrifice by cervical dislocation, the hind limbs were removed under sterile conditions and cleaned of all muscular and connective tissue. Bone marrow from the femoral and tibial cavities was flushed out with Dulbecco's Modified Eagle's medium supplemented with 10% heat inactivated fetal calf serum, 2 mM L-glutamine, 100 U/ml penicillin, and 100 mg/ml streptomycin sulfate (Gibco BRL, Gaithersburg, Md.). The bone marrow was separated into a single cell suspension by sequential passage through 21, 24, and 26 gauge needles. The remaining long bones then are crushed mechanically to a fine suspension, using sterile scissors, and further degraded into a fine powder by sequential passage through a 16- and then an 18-gauge needle such as those sold by Becton Dickinson and Company (Franklin Lakes, N.J.). Both the bone and marrow components were combined with the delivery vehicle in vitro prior to in vivo implantation.

### EXAMPLE 2

Preparation of Delivery Vehicles: Neutralized collagen extracellular matrix was prepared by combining 600 μl of type I rat tail collagen (3.69 mg/ml) (Collaborative Biomedical, Bedford, Mass.) with 100 μl of sterile 0.1N NaOH, 350 μl of 3×Dulbecco's Modified Eagle's Medium and 525 μl of 1×Dulbecco's Modified Eagle's medium with 30% fetal calf serum. Diluted Matrigel® basement membrane matrix, growth factor-reduced and phenol red-free), a product of Collaborative Biomedical (Bedford, Mass.), was prepared by combining 400 μl of Matrigel® with 200 μl of 3×Dulbecco's Modified Eagle's Medium supplemented with 30% fetal calf serum. Both mixtures were kept on ice until implantation. A 1 mm thick sheet of 95% porous, non-woven polyglycolic acid mesh (PGA) (Davis & Geck) was cut into pieces approximately 0.25 cm² and three free edges of two adjacent pieces were sewn together in order to create a bilayer pocket. The mesh was sterilized by a four-hour submersion in 100% isopropyl alcohol and overnight exposure to UV radiation in a tissue culture hood. The construct was washed several times in sterile Dulbecco's Modified Eagle's medium prior to implantation.

### EXAMPLE 3

Implantation Procedure: The cell suspension obtained from the mechanical degradation of one to two hind limbs was combined with 500 μl of a hydrogel delivery vehicle consisting of either neutralized collagen matrix or the diluted Matrigel® basement matrix in a tuberculin syringe. By injecting an identical volume of the cell suspension between the two leaves of a polyglycolic acid construct (PGA) and oversewing the remaining open edge a surgically implantable biocompatible construct was manufactured for implantation. Due to the gelatinous nature of the crushed bone/bone marrow suspension, minimal cell loss occurred through the interstices of the PGA mesh prior to transplantation. All constructs were kept on ice until implantation. Recipient animals consisted of syngeneic C57/BL6 mice ranging between 12 and 30 weeks of age. Prior to both injection and implantation, the animals were anesthetized with Metofane™, a product of Mallinckrodt Veterinary Inc. (Mundelein, Ill.), and the surgical site was prepped with betadine. Only one site per animal was used for cell transplantation.

Subcutaneous injection of the collagen based construct into the left flank was done using a tuberculin syringe capped with a 16 gauge needle while the polyglycolic acid mesh based construct was surgically implanted into a subcutaneous pocket created by blunt dissection in the same location. Intramuscular injection was carried out in a similar manner after exposure of the rectus abdominis muscle through a midline skin incision. Implantation of the PGA construct within the rectus abdominis musculature was accomplished by creating a pocket between the muscle layers. The area of injection or construct implantation was marked by 7-0 polypropylene sutures.

For intramesenteric implantation a midline celiotomy was performed and the small bowel was eviscerated. After exposure of the mesentery down to its root, folding two leaves of the mesentery upon one another and securing them together with a 7-0 polypropylene suture created an intramesenteric pocket. In separate sets of experiments type I collagen and Matrigel® based constructs were injected into this pocket and allowed to harden prior to abdominal closure. The PGA based construct was surgically implanted into this location using a 7-0 polypropylene suture. Animals were allowed to recover with free access to food and water prior to analysis.

### EXAMPLE 4

Morphologic Analysis: Animals in each experimental group were sacrificed at an early (3 to 6 weeks) or a delayed time point (6-12 weeks) after implantation. Results are based on two to four animals analyzed per experimental group. After sacrifice by cervical dislocation, the area of cellular implantation was excised, fixed in 10% buffered formalin, decalcified in Cal-Ex (Fisher Scientific, Pittsburgh, Pa.) and embedded in paraffin. Five-micron thick sections from representative areas were prepared and stained with hematoxylin and eosin. The slides were reviewed by light microscopy for cellular engraftment, the presence of bone marrow, and overall inflammatory response.

### EXAMPLE 5

Immunohistochemistry: To further define the hematopoietic elements in the tissue engineered bone marrow immunohistochemical staining for hematopoietic stem cells was performed. CD 34 is a glycoprotein whose surface expression has previously been used to identify hematopoietic stem cells within bone marrow. After paraffin removal from five-micron thick sections of the tissue-engineered bone marrow, the slides were rehydrated for processing. After high temperature antigen retrieval, using Antigen Unmasking Solution (product of Vector Laboratories, Burlingame, Calif.) and appropriate blocking steps, the sections were incubated with rat anti mouse CD34 (clone RAM34), a product of Pharmingen (San Diego, Calif.) in 4° C. at a 1:10 dilution. After overnight incubation unbound antibody was washed off and the sections were incubated with biotinylated rabbit anti-rat antibody. Immunoperoxidase development was accomplished after signal amplification by Avidin: Biotinylated Enzyme Complex kit, marketed by Vector Laboratories. Positive reactivity was classified as dark brown staining with appropriate normal bone marrow sections serving as a positive control.

### EXAMPLE 6

Transmission Electron Microscopy: After processing, fixation and decalcification of bone marrow constructs, 50 μm sections were cut from the paraffin sections and rehydrated. The constructs were then post-fixed in 2% Osmium with 0.1M Sodium Cacodylate followed by enblock stain in 2% aqueous Uranyl Acetate. The tissue then was dehydrated in graded alcohol, clarified in propylene oxide and, after infiltrating with EPON, embedded in the same resin. Resin was heat cured at 70° C. for 48 hours and 70 nM thin sections were cut using a Leica Ultracut S microtome and Diatome diamond knife. Sections were picked up on 200 mesh thin bar grids, stained with saturated alcoholic Uranyl acetate, counterstained in Bismuth subnitrate and observed in JEOL JEM 1010. Images were captured using Hamamatsu CCD camera and AMT HR-12 systems.

### EXAMPLE 7

Subcutaneous injection of the cell suspension in a type I collagen gel and surgical implantation of the PGA based cellular construct into a subcutaneous pocket resulted in the formation of a palpable nodule that persisted for the duration of the experiment. Gross and histologic evaluation of specimens from two time points revealed minimal cellular survival, a devitalized bony matrix with a dense inflammatory response, and no marrow formation (FIG. 1). Injecting or surgically implanting identical cellular constructs within the highly vascular intramuscular pocket increased survival of osteocytes within lacunae of the cancellous bone but did not result in bone marrow formation (FIG. 2).

Small bowel mesentery seeded with marrow stroma in a type I collagen gel had a palpable nodule in all animals studies. Gross examination upon sectioning revealed numerous islands of bloody, gelatinous material resembling bone marrow (FIG. 3). Histologic evaluation confirmed the presence of bone marrow with distinct islands of hematopoiesis separated by blood vessels, sinusoids, and adventitial reticular cells. Upon closer examination megakaryocytes were also discernable (FIGS. 4, 5, and 6). Transmission electron microscopy revealed the presence of early hematopoietic precursors surrounding endothelial sinusoids and reticular cells (FIG. 7) and immunohistochemical evaluation revealed identifiable CD 34 positive hematopoietic stem cells within the tissue (FIG. 8). Parallel rows of rope like type I collagen fibers could also be seen interspersed within the constructs (FIG. 9). When PGA mesh rather than type I collagen gel was used as the delivery vehicle, no marrow formation was evident despite survival and engraftment of bone (FIG. 10).

### EXAMPLE 8

To test the role of type I collagen in the formation of bone marrow, Matrigel®, consisting of type IV collagen (30%) and laminin (61%), was used as the delivery vehicle to the mesenteric site in a portion of animals. No marrow formation was visible under these conditions.

What we claim is:

1. An engineered bone marrow comprising bone marrow cells, pulverized bone or bone substitute, and type I collagen or collagen substitute.

2. The engineered bone marrow according to claim 1, further comprising a hematopoietic stem cell, wherein said bone marrow cells support the maintenance and differentiation of said hematopoietic stem cell, and wherein said hematopoietic stem cell and said bone marrow cells have the same MHC haplotype.

3. The engineered bone marrow according to claim 2, comprising the complete cellular component of bone marrow obtained from a single donor.

4. The engineered bone marrow according to claim 1, comprising a bone substitute that is selected from the group consisting of a calcium phosphate, a bioactive glass, and a bioactive ceramic.

5. The engineered bone marrow according to claim 1, wherein said collagen substitute is a mammalian derived gelatin- or collagen-containing surgical sponge.

6. The engineered bone marrow according to claim 1, wherein the proportion of bone marrow cells: pulverized bone or bone substitute: collagen is about 8-98%: 1-91%: and 0.004-1.0%.

7. The engineered bone marrow according to claim 6, wherein the proportion of bone marrow cells: pulverized bone or bone substitute: collagen is about 32-91%: 9-67%: 0.01-1.0%.

8. The engineered bone marrow according to claim 7, wherein the proportion of bone marrow cells: pulverized bone or bone substitute: and collagen or collagen substitute is about 48-84%: 16-52%: 0.03-1.0%.

9. A method of making an engineered bone marrow, comprising combining about 1×10⁶ to about 5×10⁸ bone marrow cells, with about 50-500 mg of pulverized bone or bone substitute, and about 100 μl to about 750 μl of neutralized type I collagen matrix that has a collagen concentration of about 1.0 mg/ml to about 2.0 mg/ml.

10. The method according to claim 9, comprising combining about 5×10⁶ to about 1×10⁸ bone marrow cells, with about 50 mg to about 500 mg of pulverized bone or bone substitute, and about 100 μl to about 750 μl of neutralized type I collagen matrix that has a collagen concentration of about 1.0 mg/ml to about 2.0 mg/ml.

11. The method according to claim 10, comprising combining about 1×10⁷ to about 6×10⁷ bone marrow cells, with about 50 to about 500 mg of pulverized bone or bone substitute, and about 100 μl to about 750 μl of neutralized type I collagen matrix that has a collagen concentration of about 1.0 mg/ml to about 2.0 mg/ml.

12. The method according to claim 11, wherein 10×10⁶ to 20×10⁶ bone marrow cells are combined with 50 mg to about 200 mg of pulverized bone or bone substitute.

13. The method according to claim 12, wherein said bone substitute is selected from the group consisting of a calcium phosphate, a bioactive glass, and a bioactive ceramic.

14. A method of implanting an engineered bone marrow, comprising introducing said engineered bone marrow of claim 1 into a vascular site in the portal system of a mammal.

15. The method according to claim 14, wherein said engineered bone marrow is implanted in a pocket of an intramesenteric portal site.

16. The method according to claim 15, wherein said engineered bone marrow is implanted in an intramesenteric pocket of the small bowel mesentery.

17. The method according to claim 16, wherein the ratio of said volume of said engineered bone marrow implanted to the volume of said mesentery is about 0.1 to about 0.75:1.

18. The method according to claim 17, wherein the ratio of said volume of engineered bone marrow implanted to the volume of said mesentery is about 0.1 to about 0.5:1.
